(19) United States
(12) Patent Application Publication
(10) Pub. No.: US 2023/0363812 A1
(43) Pub. Date: Nov. 16, 2023

(54) THERMAL CUTTER ASSEMBLY AND SEAL PLATE ASSEMBLY AND METHOD FOR MANUFACTURING SAME

(71) Applicant: Covidien LP, Mansfield, MA (US)

(72) Inventors: Jake H. Young, Erie, CO (US); Craig V. Krastins, Arvada, CO (US); James D. Allen, IV, Broomfield, CO (US)

(21) Appl. No.: 18/197,839

(22) Filed: May 16, 2023

Related U.S. Application Data
(60) Provisional application No. 63/342,171, filed on May 16, 2022.

(51) Int. Cl. A61B 18/08

(52) U.S. Cl. CPC A61B 18/085; A61B 2018/00404

(57) ABSTRACT

A jaw member for an end effector assembly of a vessel sealing instrument includes a seal plate assembly having first and second seal plates joined atop one another, the second seal plate defining a channel extending from a proximal to a distal end thereof. A thermal cutter assembly includes a substrate disposed within the channel which extends from the proximal to the distal end of the second seal plate. An insulator is disposed atop the substrate and is configured to extend therealong. A resistive element is disposed atop the insulator and is configured to generate heat upon activation thereof. An encapsulant is configured to electrically insulate the resistive element and thermally conduct heat from the resistive element, such that, upon activation thereof, tissue disposed within the end effector assembly is cut along the resistive element.

### CROSS REFERENCE TO RELATED APPLICATION

This application claims the benefit of and priority to U.S. Provisional Patent Application No. 63/342,171, filed on May 16, 2022, the entire contents of which are hereby incorporated herein by reference.

### FIELD

The present disclosure relates to surgical instruments and, more particularly, to electrosurgical instruments for sealing and cutting tissue, and methods of manufacturing same.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy-based tissue cutting element may be provided to cut the treated tissue using energy, e.g., thermal, electrosurgical, ultrasonic, light, or other suitable energy.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is a jaw member for an end effector assembly of a vessel sealing instrument which includes a seal plate assembly having first and second seal plates joined atop one another, the second seal plate defining a channel extending from a proximal to a distal end thereof. A thermal cutter assembly includes: a substrate disposed within the channel and extending from the proximal to distal end of the second seal plate; an insulator disposed atop the substrate and configured to extend therealong; a resistive element disposed atop the insulator and configured to generate heat upon activation thereof; and an encapsulant configured to electrically insulate the resistive element and thermally conduct heat from the resistive element, such that, upon activation thereof, tissue disposed within the end effector assembly is cut along the resistive element.

In aspects in accordance with the present disclosure, the substrate is made from a material having a low thermal conductivity.

In aspects in accordance with the present disclosure, the substrate is disposed within the channel utilizing a thermal spraying or masking process.

In aspects in accordance with the present disclosure, the insulator is disposed atop the substrate using a deposition process.

In aspects in accordance with the present disclosure, the insulator is made from a material having a high coefficient of thermal conductivity but low electrical conductivity. In other aspects in accordance with the present disclosure, the insulator is treated after the deposition process to facilitate adhesion of the resistive element thereon.

In aspects in accordance with the present disclosure, opposing ends of the resistive element are configured to connect to a pair of conductive pads disposed at a proximal end of the insulator.

In aspects in accordance with the present disclosure, the encapsulant is made from an electrically insulative, highly thermally conductive material.

Provided in accordance with aspects of the present disclosure is an end effector assembly of a vessel sealing instrument which includes first and second jaw members movable between a spaced apart position and an approximated position for sealing and cutting tissue, the first jaw member including: a first seal plate assembly including first and second seal plates joined atop one another, the second seal plate defining a first channel extending from a proximal to a distal end thereof. A thermal cutter assembly is included having: a first substrate disposed within the first channel and extending from the proximal to distal end of the second seal plate; an insulator disposed atop the first substrate and configured to extend therealong; a resistive element disposed atop the insulator and configured to generate heat upon activation thereof and an encapsulant configured to electrically insulate the resistive element and thermally conduct heat from the resistive element, such that, upon activation thereof, tissue disposed between opposing first and second jaw members of the end effector assembly is cut along the resistive element. The second seal plate includes a second seal plate assembly including first and second seal plates joined atop one another, the second seal plate defining a second channel extending from a proximal to a distal end thereof configured to receive a second substrate therein, wherein the second substrate opposes the thermal cutter assembly of the first jaw member when the first and second jaw members are moved to the approximated position.

In aspects in accordance with the present disclosure, the first and second substrates are made from materials having a low thermal conductivity.

In aspects in accordance with the present disclosure, one or both of the first and second substrates is disposed within a respective first and second channel utilizing a thermal spraying or masking process.

In aspects in accordance with the present disclosure, the insulator is disposed atop the first substrate using a deposition process.

In aspects in accordance with the present disclosure, the insulator is made from a material having a high coefficient of thermal conductivity but low electrical conductivity. In other aspects in accordance with the present disclosure, the insulator is treated after the deposition process to facilitate adhesion of the resistive element thereon.

In aspects in accordance with the present disclosure, opposing ends of the resistive element are configured to connect to a pair of conductive pads disposed at a proximal end of the insulator.

In aspects in accordance with the present disclosure, the encapsulant is made from an electrically insulative, highly thermally conductive material.

Provided in accordance with aspects of the present disclosure is a method of manufacturing a jaw member of an end effector assembly of a vessel sealing instrument which includes joining first and second seal plates to form a seal plate assembly, the second seal plate defining a channel extending from a proximal to a distal end thereof. The channel configured to support a thermal cutter assembly formed therein via: disposing a substrate within the channel extending from the proximal to distal end of the second seal plate; disposing an insulator atop the substrate and extending the insulator therealong; disposing a resistive element atop the insulator, the resistive element configured to generate heat upon activation thereof; and encapsulating the resistive element with an electrically insulative, thermally conductive material, such that, upon activation of the resistive element, the electrically insulative, thermally conductive material heats to a temperature to cut tissue.

In aspects in accordance with the present disclosure, the substrate is made from a material having a low thermal conductivity and is disposed within the channel by a thermal spraying or masking process.

In aspects in accordance with the present disclosure, the insulator is made from a material having a high coefficient of thermal conductivity but low electrical conductivity and is disposed atop the substrate using a deposition process.

In aspects in accordance with the present disclosure, disposing the resistive element atop the insulator includes sputtering or thick film printing.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure shown connected to an electrosurgical generator;

FIG. 2 is a perspective view of a hemostat-style electrosurgical forceps provided in accordance with the present disclosure;

FIG. 3 is a schematic illustration of a robotic surgical instrument provided in accordance with the present disclosure;

FIG. 4 is a perspective view of a distal end portion of the forceps of FIG. 1, wherein first and second jaw members of an end effector assembly of the forceps are disposed in a spaced-apart position exposing a thermal cutter assembly;

FIG. 5 is a perspective view of a distal end portion of the forceps of FIG. 1, wherein first and second jaw members of the end effector assembly of the forceps are disposed in a spaced-apart position and the thermal cutter assembly is separated therefrom exposing a slot defined in the second jaw member;

FIG. 6A is a schematic view of the thermal cutter assembly in accordance with the present disclosure;

FIG. 6B is a schematic side view of the thermal cutter assembly in accordance with the present disclosure;

FIG. 7A is a perspective view of the second jaw member including a seal plate assembly and thermal cutter assembly manufactured in accordance with another embodiment of the present disclosure;

FIG. 7B is a perspective view of the seal plate assembly and thermal cutter assembly of FIG. 7A;

FIGS. 8A-8E are perspective views showing the various assembly steps for both the seal plate assembly and the thermal cutter assembly of FIG. 7A;

FIG. 9A is an exploded view of the second jaw member of FIG. 7A;

FIG. 9B is a front cross-sectional view of the second jaw member of FIG. 7A taken along line 9B-9B; and

FIG. 10 is a bottom, perspective view of the first jaw member including a substrate disposed in vertical opposition of the thermal cutter assembly of the second jaw member.

### DETAILED DESCRIPTION

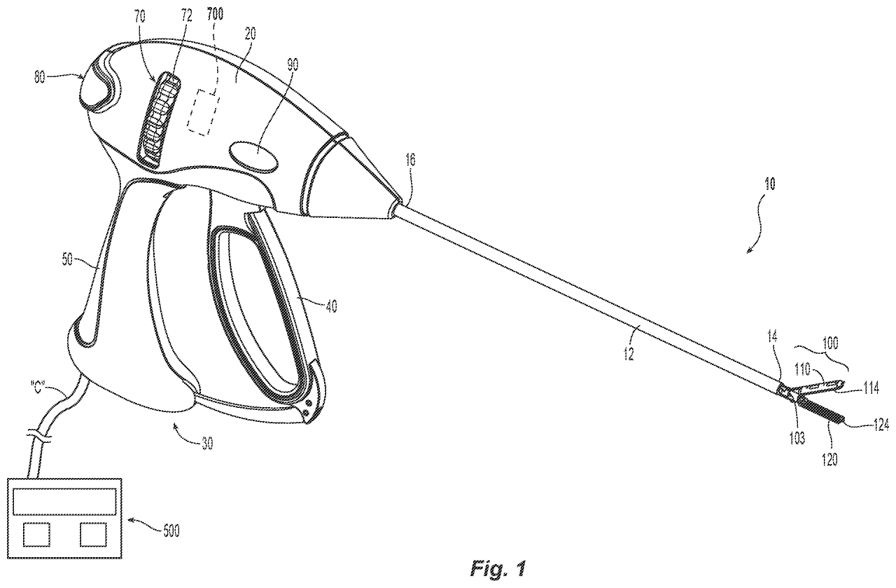

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

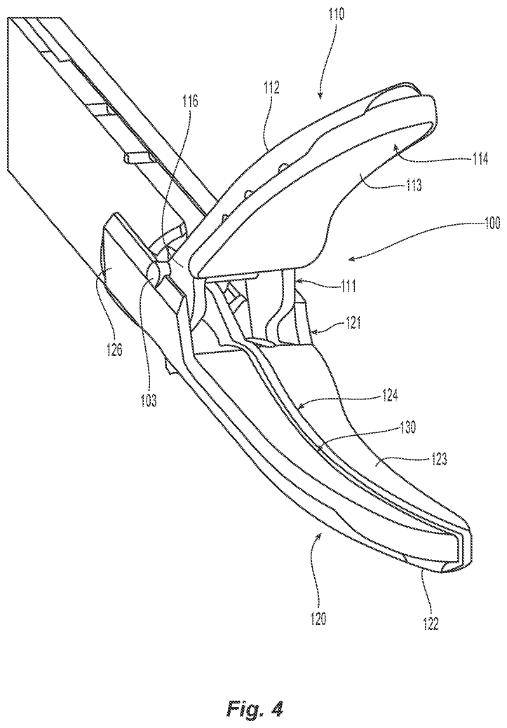

Forceps 10 includes a housing 20, a handle assembly 30, a rotating assembly 70, a first activation switch 80, a second activation switch 90, and an end effector assembly 100. As shown, end effector assembly 100 includes jaw members 110 and 120 configured for unilateral movement relative to one another. Bilateral movement of the jaw members 110, 120 is also envisioned. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable “C” that connects forceps 10 to an energy source, e.g., an electrosurgical generator “G.” Cable “C” includes a wire (or wires) (not shown) extending therethrough that has sufficient length to extend through shaft 12 in order to connect to one or both tissue-treating surfaces 114, 124 of jaw members 110, 120, respectively, of end effector assembly 100 (see FIG. 4) to provide energy thereto.

First activation switch 80 is coupled to tissue-treating surfaces 114, 124 (FIG. 4) and the electrosurgical generator “G” for enabling the selective activation of the supply of energy to jaw members 110, 120 for treating, e.g., cauterizing, coagulating/desiccating, and/or sealing, tissue. Second activation switch (e.g., thumb switch 90) is coupled to thermal cutter assembly 130 of jaw member 120 (FIG. 4) and the electrosurgical generator “G” for enabling the selective activation of the supply of energy to thermal cutter assembly 130 for thermally cutting tissue. Second activation switch 90 may be actuated via any finger, in-line with handle, footswitch, etc.

Alternatively, a single activation switch may be utilized wherein the generator “G” sequentially seals and then cuts with a single actuation of the switch, e.g., switch 80. A “seal” may be indicated by an audible tone from the generator “G” and after a short or programmable delay the forceps 10 (or the generator algorithm) transitions into a cut cycle or cut “mode”. Again a “cut” may be represented by a different tone from the generator “G” or from the forceps 10.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position and an approximated position to grasp tissue between tissue-treating surfaces 114, 124 of jaw members 110, 120. As shown in FIG. 1, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120. Rotating assembly 70 includes a rotation wheel 72 that is selectively rotatable in either direction to correspondingly rotate end effector assembly 100 relative to housing 20.

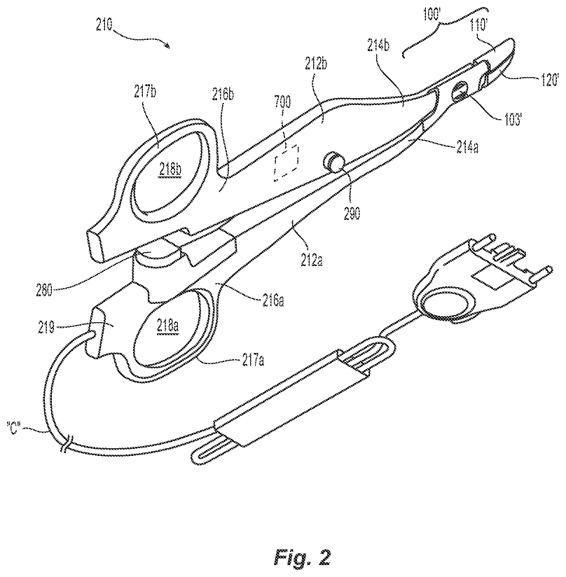

Referring to FIG. 2, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to end effector assembly 100 (FIG. 4). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to a source of energy, e.g., electrosurgical generator “G” (FIG. 1). Proximal shaft connector 219 secures a cable “C” to forceps 210 such that the user may selectively supply energy to jaw members 110′, 120′ for treating tissue. More specifically, a first activation switch 280 (similar to activation switch 80 discussed above) is provided for supplying energy to jaw members 110′, 120′ to treat tissue upon sufficient approximation of shaft members 212a, 212b, e.g., upon activation of first activation switch 280 via shaft member 212a. A second activation switch 290 (similar to second activation switch 90 discussed above) disposed on either or both of shaft members 212a, 212b is coupled to the thermal cutter element (not shown, similar to thermal cutter assembly 130 of jaw member 120 (FIG. 4)) of one of the jaw members 110′, 120′ of end effector assembly 100′ and to the electrosurgical generator “G” for enabling the selective activation of the supply of energy to the thermal cutter assembly 130 for thermally cutting tissue.

Alternatively, a single activation switch may be utilized wherein the generator “G” sequentially seals and then cuts with a single actuation of the switch, e.g., switch 280. A “seal” may be indicated by an audible tone from the generator “G” and after a short or programmable delay the forceps 210 (or the generator algorithm) transitions into a cut cycle or cut “mode”. Again a “cut” may be represented by a different tone from the generator “G” or from the forceps 210.

Jaw members 110′, 120′ define a curved configuration wherein each jaw member is similarly curved laterally relative to a longitudinal axis of end effector assembly 100′. However, other suitable curved configurations including curvature towards one of the jaw members 110, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

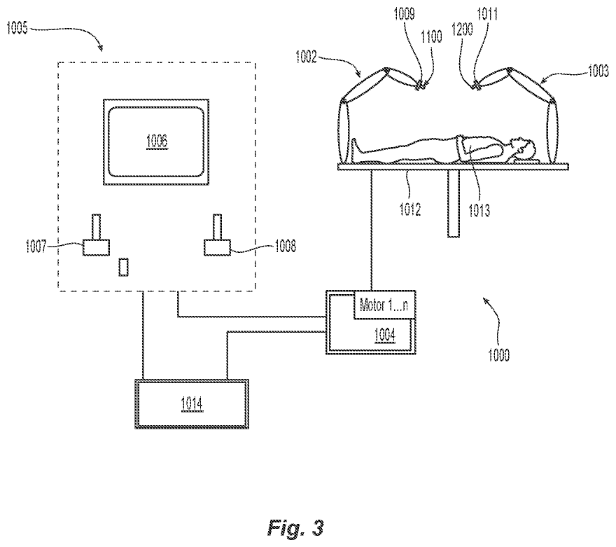

Referring to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical instrument 1000 may be configured for use on a patient 1013 lying on a patient table 1012 to be treated in a minimally invasive manner. Robotic surgical instrument 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to end effector assembly 100 (FIG. 4), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

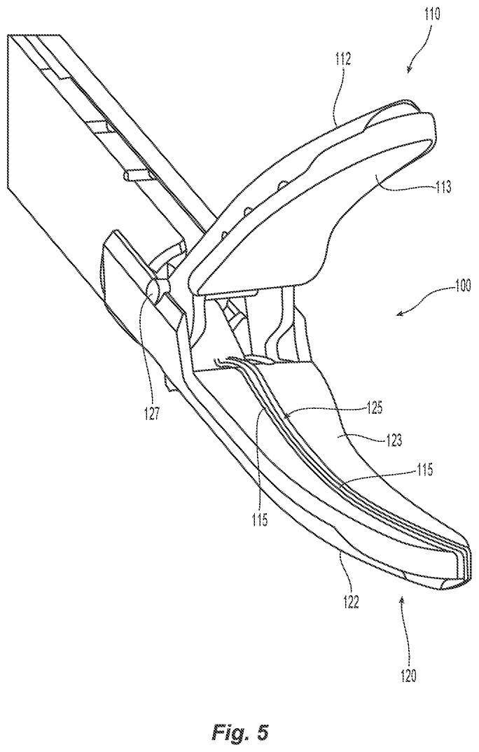

Turning to FIGS. 4-5, end effector assembly 100, as noted above, includes first and second jaw members 110, 120. Each jaw member 110, 120 may include a structural frame 111, 121, a jaw housing 112, 122, and a tissue-treating plate 113, 123 defining the respective tissue-treating surface 114, 124 thereof. Alternatively, only one of the jaw members, e.g., jaw member 120, may include structural frame 121, jaw housing 122, and tissue-treating plate 123 defining the tissue-treating surface 124. In such embodiments, the other jaw member, e.g., jaw member 110, may be formed as a single unitary body, e.g., a piece of conductive material acting as the structural frame 111 and jaw housing 112 and defining the tissue-treating surface 114.

An outer surface of the jaw housing 112, in such embodiments, may be at least partially coated with an electrically insulative material or may remain exposed. In embodiments, tissue-treating plates 113, 123 may be deposited onto jaw housings 112, 122 or jaw inserts (not shown) disposed within jaw housings 112, 122, e.g., via sputtering. Alternatively, tissue-treating plates 113, 123 may be pre-formed and engaged with jaw housings 112, 122 and/or jaw inserts (not shown) disposed within jaw housings 112, 122 via, for example, overmolding, adhesion, mechanical engagement, etc. Other methods of depositing the tissue-treating plates 113, 123 onto the jaw inserts are described in detail below.

Referring in particular to FIGS. 4 and 5, jaw member 110, as noted above, may be configured similarly as jaw member 120, may be formed as a single unitary body, or may be formed in any other suitable manner so as to define a structural frame 111 and a tissue-treating surface 114 opposing tissue-treating surface 124 of jaw member 120. Structural frame 111 includes a proximal flange portion 116 about which jaw member 110 is pivotably coupled to jaw member 120. In shaft-based or robotic embodiments, proximal flange portion 116 receives pivot 103 and which mounts atop flange 126 of jaw member 120 (FIG. 4) such that actuation of movable handle 40 (FIG. 1) or a robotic drive, pivots jaw member 110 about pivot 103 and relative to jaw member 120 between the spaced-apart position and the approximated position. However, other suitable drive arrangements are also contemplated, e.g., using cam pins and cam slots, a screw-drive mechanism, etc.

For the purposes of further describing one or both of the jaw members 110, 120 (and 210, 220), each jaw member 110, 120 may include a longitudinally-extending insulative member 115 defined within a slot 125 extending along at least a portion of the length of tissue-treating surfaces 114, 124 (FIG. 5). Insulative member 115 may be transversely centered on either or both tissue-treating surfaces 114, 124 or may be offset relative thereto. As explained in more detail below with respect to jaw member 120, insulative member 115 may house and electrically and/or thermally isolate the thermal cutter assembly 130 separately activatable to cut tissue upon activation thereof. Further, insulative member 115 may be disposed, e.g., deposited, coated, etc., on tissue-treating surface 114, 124, may be positioned within the channel or recess defined within tissue-treating surface 114, 124, or may define any other suitable configuration.

Additionally, insulative member 115 may be substantially (within manufacturing, material, and/or use tolerances) coplanar with each respective tissue-treating surface 114, 124 may protrude from each respective tissue-treating surface 114, 124, may be recessed relative to each respective tissue-treating surface 114, 124 or may include different portions that are coplanar, protruding, and/or recessed relative to tissue-treating surfaces 114, 124. Moreover, insulative member 115 and thermal cutter assembly 130 may be curvilinear to follow the configuration of the jaw members 110, 120. Insulative member 115 may be formed from, for example, ceramic, parylene, glass, nylon, PTFE, or other suitable material(s) (including combinations of insulative and non-insulative materials).

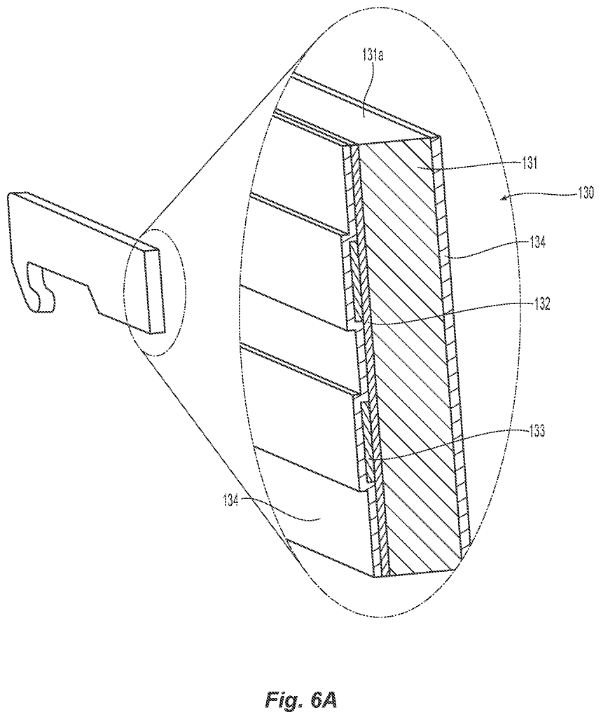

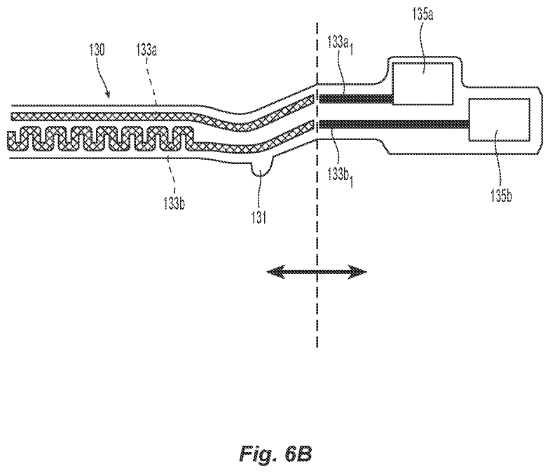

With reference to FIGS. 4 and 5, as noted above, jaw member 120 includes a structural frame 121, a jaw housing 122, and a tissue-treating plate 123 defining the tissue-treating surface 124 thereof. With reference also to FIG. 6A, details relating to the thermal cutter assembly 130 are generally defined to include the following elements (described internally to externally): substrate 131 or other internalized bendable metal structure that is both thermally and electrically conductive, e.g., stainless steel, aluminum, etc.; insulator 132 having generally electrically insulative properties and at least partially conductive, e.g., sintered glass, alumina, Poly Ethylene Oxide (PEO), Silica, etc.; resistive element 133 or any metal that is resistive but certain metals may have better thermal coefficients than others; and encapsulant 134 or an electrically insulative materials that is at least partially thermally conductive (may be the same or similar to the insulator). As explained below, the resistive element 133 may be deposited atop insulator 132 via sputtering or the like.

FIG. 6B shows a side view of thermal cutter assembly 130 and the electrical connections associated therewith. Generally, electrically conductive pads 135a, 135b connect to opposite ends 133a, 133b of resistive element 133 via traces 133a1, 133b1 which are electrically conductive traces (low resistance/low heat). As explained in detail below, resistive element 133 is configured to rapidly generate heat due to high resistive properties when electrical current is passed therethrough.

Structural frame 121 defines a proximal flange portion 126 and a distal body portion (not shown) extending distally from proximal flange portion 126. Proximal flange portion 126 is bifurcated to define a pair of spaced-apart proximal flange portion segments that receive proximal flange 111 of jaw member 110 therebetween and define aligned apertures 127 configured for receipt of pivot 103 therethrough/thereon to pivotably couple jaw members 110, 120 with one another (FIG. 5).

Jaw housing 122 of jaw member 120 is disposed about the distal body portion of structural frame 121, e.g., via overmolding, adhesion, mechanical engagement, etc., and supports tissue-treating plate 123 thereon, e.g., via overmolding, adhesion, mechanical engagement, depositing (such as, for example, via sputtering or thermal spraying), etc. Tissue-treating plate 123, as noted above, defines tissue-treating surface 124. Longitudinally-extending slot or channel 125 is defined through tissue-treating plate 123 and is positioned relative to jaw member 110 or an insulative member 115 disposed in vertical registration therewith when the jaw members 110 and 120 are in the approximated position (FIG. 5). The slot or channel 125 may be defined within an integrally-formed tissue-treating plate 123 or may be defined between two tissue-treating plates that, together, operate as a single treatment surface (not shown). Slot 125 may extend through at least a portion of jaw housing 122, a jaw insert (if so provided), and/or other components of jaw member 120 to enable receipt of thermal cutter assembly 130 at least partially within slot 125.

Thermal cutter assembly 130, more specifically, is disposed within longitudinally-extending slot 125 such that thermal cutter assembly 130 opposes jaw member 110 in the approximated position. Thermal cutter assembly 130 may be configured to contact jaw member 110 (or another insulative member 115 as mentioned above and as shown in FIG. 4) in the approximated position to regulate or contribute to regulation of a gap distance between tissue-treating surfaces 114, 124 in the approximated position. Alternatively or additionally, one or more stop members (not shown) associated with jaw member 110 and/or jaw member 120 may be provided to regulate the gap distance between tissue-treating surfaces 114, 124 in the approximated position.

Thermal cutter assembly 130 may be surrounded by the insulative member 115 disposed within slot 125 to electrically and/or thermally isolate thermal cutter assembly 130 from tissue-treating plate 123 (See FIG. 4 versus FIG. 5). As mentioned above, thermal cutter assembly 130 includes an encapsulant 134 that may act in conjunction with or in lieu of insulative member 115. Encapsulant 134 (and insulator 132 as shown in FIG. 6A) is configured cover the sides of the substrate 131 leaving the tissue facing edge 131a of the substrate 131 exposed. Thermal cutter assembly 130 and insulative member 115 may similarly or differently be substantially (within manufacturing, material, and/or use tolerances) coplanar with tissue-treating surface 124, may protrude from tissue-treating surface 124, may be recessed relative to tissue-treating surface 124, or may include different portions that are coplanar, protruding, and/or recessed relative to tissue-treating surface 124.

Turning back to the thermal cutter assembly 130 and the various methods of manufacturing the same, it is contemplated that the resistive element 133 of the thermal cutter assembly 130 may be manufactured in thin layers that are deposited atop (or otherwise) insulator 132 which is disposed atop substrate 131. For the purposes herein, the resistive element 133 will be described as being deposited onto insulator 132, knowing that insulator, in turn, may be disposed on one or both sides of substrate 131. For example, it is contemplated that resistive element 133 may be deposited onto the insulator 132 via one or more of the following manufacturing techniques: sputtering, thermal evaporation, thermal spraying, cathodic arcing, pulsed laser deposition, electron beam deposition. Other techniques may include: electroless strike or plating and electro-plating, shadow masking.

Utilizing one or more of these techniques provides a thin layer of resistive material which has the benefit of dissipating heat quickly compared to a traditional thermal cutter assembly 130. Other advantages of thin-layered resistive elements 133 on the thermal cutter assembly 130 include: the ability to heat up quickly, the ability to require less energy to heat up and maintain heat during the cutting process, and the ability to cut tissue in a reduced timeframe compared to traditional electrical cutters.

Any one of the following materials (or combinations thereof) may be utilized as the resistive element 133: aluminum, copper, chromium, titanium, stainless steel, nickel, chrome, tin, platinum, palladium, gold, nichrome, and Kanthal®. It is contemplated that during manufacturing, combinations of materials may be utilized for a particular purpose or to achieve a particular result. For example, one material may be utilized as a base conductor with a second material used as an outer or inner conductor to act as the heating element. Additional techniques or materials may be added to act as thermal cutter assemblies 130 or resistive elements 133 such as those described with reference to U.S. Patent Publication No. 2021/0244465 filed Feb. 7, 2020, U.S. Provisional Patent Application Ser. No. 62/952,232 filed Dec. 21, 2019, U.S. Patent Publication No. 2021/0307812A1 filed Apr. 2, 2020, and U.S. Patent Publication No. 2021/022798 filed Jul. 22, 2019, the entire contents of each of which being incorporated by reference herein.

In other embodiments, materials may be mixed during the application process. In some embodiments, the material used (e.g., Aluminum, copper etc.) may be thin and still promote a good cutting effect while other materials may have to be thicker to produce the same or similar cutting effect due to the particular material's level of electrical resistance. In this latter instance, a highly conductive base material may be utilized with the thinner, less conductive material more resistive material to produce a desired effect.

In embodiments, a biocompatible material (not shown) may be utilized to cover a non-biocompatible material. In other embodiments, the materials may be deposited (or otherwise disposed on insulator 132 in non-uniform layers while still allowing for transitions, e.g., side-to-side transitions. The materials could be deposited (or otherwise disposed on insulator 132) in an alternating fashion and more than one electrical circuit may be employed.

Examples of resistive elements 133 that may be used for thermal cutter assemblies 130 may include single layer resistive elements 133 in the range of about 0.1 micron to about 500 microns. A so-called “thick” film resistive element 133 would be about 30 microns and a “thin” film resistive element 133 would be about 1 micron. Non-conductive, electrically transparent, thermally transparent, or electrically and/or thermally porous materials may also be layered in a similar fashion atop, below or between the resistive elements 133. One or more of these materials may be layered atop the resistive elements 133 to complete the thermal cutter assembly 130 as mentioned above within a specified range.

Generally, tissue-treating plates 113, 123 are formed from an electrically conductive material, e.g., for conducting electrical energy therebetween for treating tissue, although tissue-treating plates 113, 123 may alternatively be configured to conduct any suitable energy, e.g., thermal, microwave, light, ultrasonic, etc., through tissue grasped therebetween for energy-based tissue treatment. As mentioned above, tissue-treating plates 113, 123 are coupled to activation switch 80 and electrosurgical generator “G” (FIG. 1) such that energy may be selectively supplied to tissue-treating plates 113, 123 and conducted therebetween and through tissue disposed between jaw members 110, 120 to treat tissue, e.g., seal tissue on either side and extending across thermal cutter assembly 130.

Thermal cutter assembly 130, on the other hand, is configured to connect to electrosurgical generator “G” (FIG. 1) and second activation switch 90 to enable selective activation of the supply of energy to thermal cutter assembly 130 for heating resistive element 133 which, in turn, heats edge 131a of substrate 131 to thermally cut tissue disposed between jaw members 110, 120, e.g., to cut the sealed tissue into first and second sealed tissue portions. Other configurations including multi-mode switches, other separate switches, etc. may alternatively be provided.

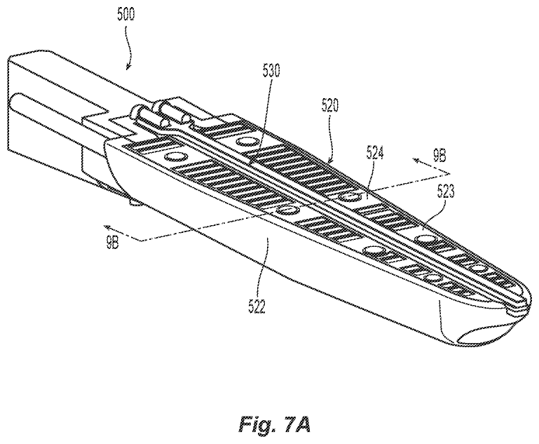

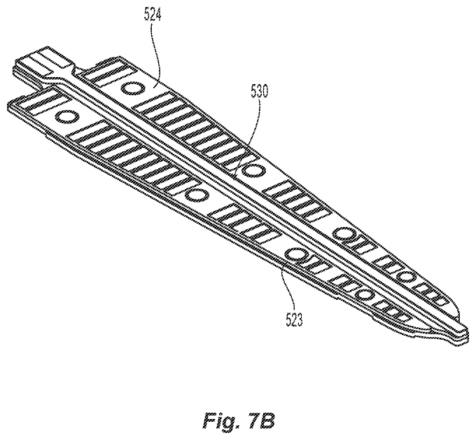

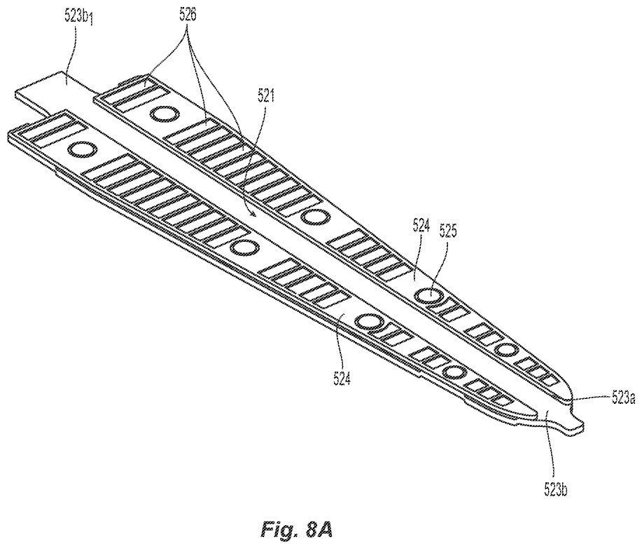

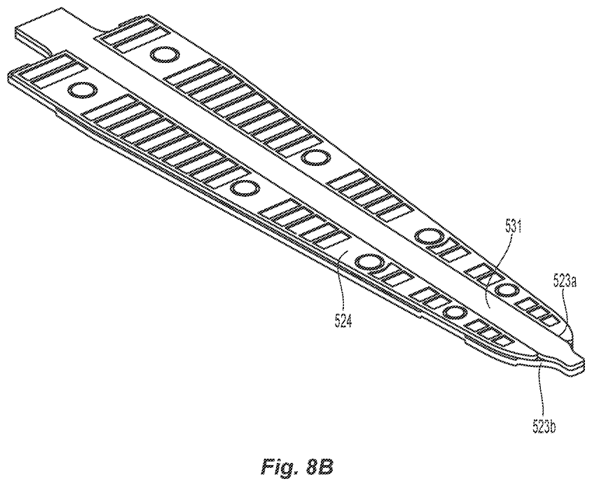

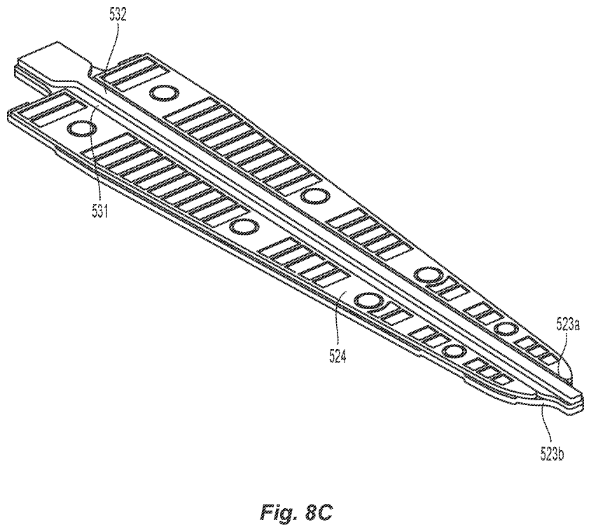

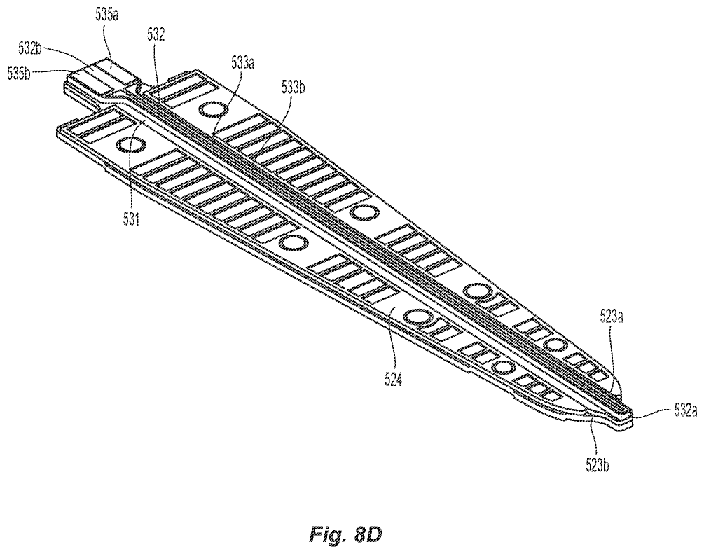

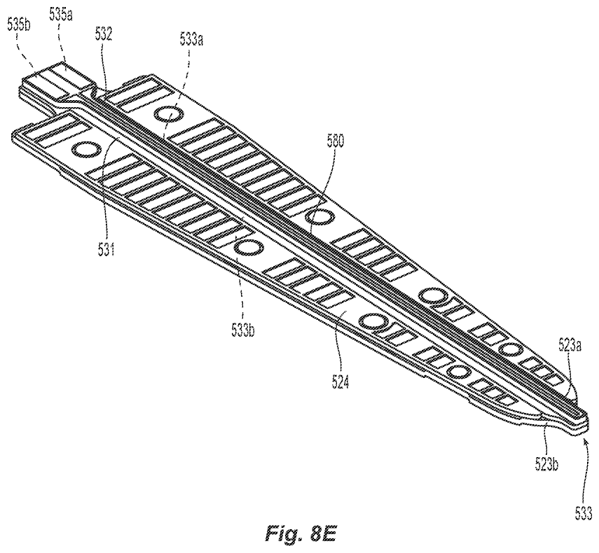

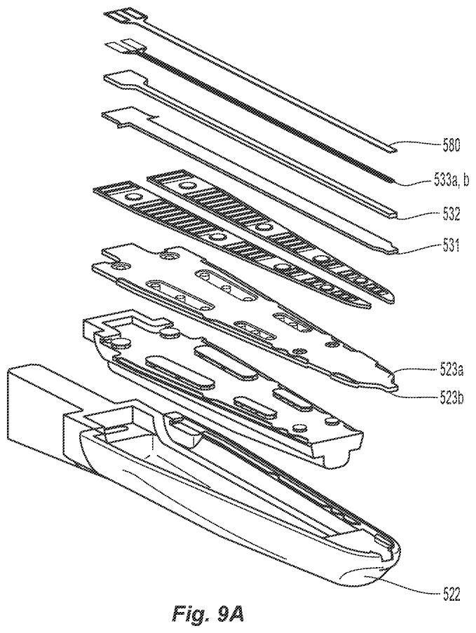

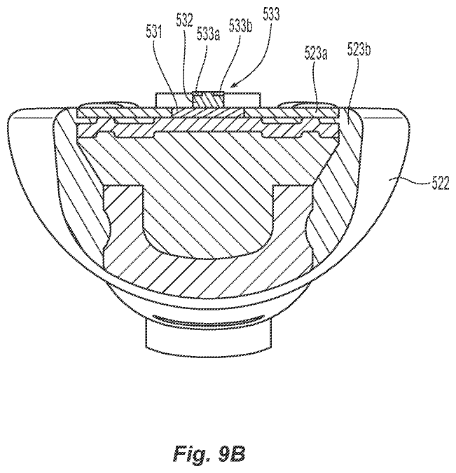

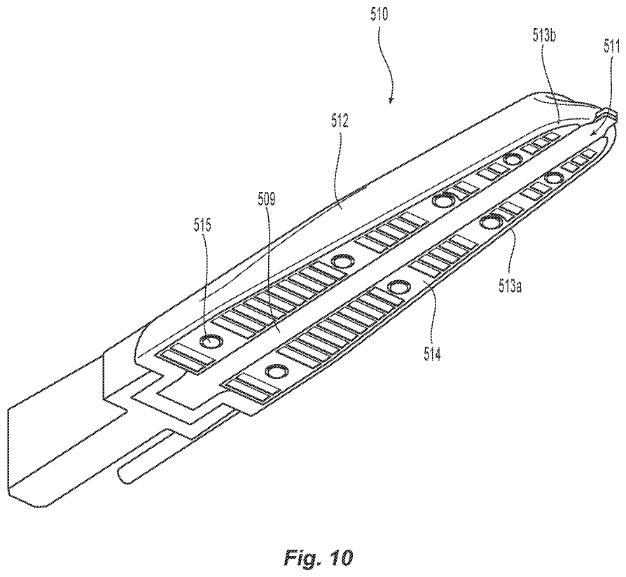

FIGS. 7A-7B show one embodiment of a jaw member 520 of an end effector assembly 500 for use with any of the aforementioned forceps 10, 210 described herein that utilizes a combination thermal cutter assembly 530 and seal plate assembly 523 that are manufactured for assembly as a unit during construction of the jaw member 520 as shown in FIGS. 9A and 9B. More particularly, FIG. 7A shows an assembled jaw member 520 and FIG. 7B shows the seal plate assembly 523 and thermal cutter assembly 530 in stand alone fashion. Details relating to the manufacture of the seal plate assembly 523 and the thermal cutter assembly 530 are shown in FIGS. 8A-8E.

As shown initially in FIG. 8A, seal plate 523a is welded atop seal plate 523b such that upper tissue contacting surface 524 extends on either side thereof defining channel 521 therebetween. Alternatively, a single seal plate, e.g., seal plate 523a, may be utilized and a channel 521 may be coined or defined therein such that upper tissue contacting surface 524 extends on either side thereof. A series of stop members 525 may be disposed atop tissue contacting surface 524 and configured to define a gap distance between opposing jaw members, e.g., jaw members 110, 120, when approximating tissue during sealing. The stop members 525 may be applied as part of a thermal spraying or other deposition process. In addition, any number of or type of gripping surfaces 526 may be defined within tissue contacting surface 524 depending upon a particular purpose. A proximal end 523b1 of seal plate 523b is configured to extend proximally relative to seal plate 523a, the purposes of which being explained in detail below.

FIG. 8B shows a substrate 531 disposed atop seal plate 523b within channel 521 and dimensioned to substantially match the dimensions of the channel 521 as shown in FIG. 8A such that a proximal end of the substrate 531 is supported on proximal end 523b′ of plate 523b. Yttria-stabilized zirconia (YSZ) (or some other type of ceramic or other type of material with a low thermal conductivity) may be utilized as a base substrate 531 for the thermal cutter assembly 530. YSZ may be deposited atop sealing plate 523b utilizing a thermal spraying or masking process.

An insulator 532, e.g., alumina or some other type of material having a high coefficient of thermal conductivity but low electrical conductivity (electrical insulator) is disposed atop substrate 531 and extends therealong to a distal end of substrate 531 (FIG. 8C). Various deposition techniques may be employed to deposit the insulator 532 atop the substrate 531, e.g., atomic layer deposition. The insulator 532 may need to be prepped or treated after deposition, e.g., post deposition, to facilitate/enhance the processing or adhesion of the next layer of the thermal cutter assembly 530, e.g., resistive element 533, thereon.

Conductive pads 535a and 535b are disposed atop insulator 532 near the proximal end 532b thereof and resistive elements 533a, 533b are configured to extend therefrom atop insulator 532. More particularly, resistive element 533a extends from pad 535a towards a distal end 532a of the insulator 532 and resistive element 533b continues the current loop around the distal end 532a and back to conductive pad 535b to close the current loop. Resistive elements 533a, 533b may include more resistive or thermally conductive portions that heat up and extend along the jaw member 520 (e.g., similar to a hot knife) and more electrically conductive, less resistive portions proximal the conductive pads 535a, 535b similar to elements 133a1, 133b1 described with respect to FIG. 6B. As mentioned above with respect to the previously described resistive elements, resistive elements 533a, 533b may be sputtered or thick film printed onto insulator 532. In addition, similar ranges for the layers of the thicknesses of the conductive materials utilized for the resistive elements 533a, 533b apply, e.g., thick film resistive element 533a, 533b would be about 30 microns and a thin film resistive element 533a, 533b would be about 1 micron.

An encapsulant 580 is applied over the resistive elements 533a, 533b (and may be applied over the conductive pads 535a, 535b (if warranted and depending upon a particular purpose) to electrically isolate the resistive elements 533a, 533b from the other parts of the thermal cutter assembly 530. Glass or other types of encapsulants that are electrically insulative and highly thermally conductive may be utilized for this purpose and may be applied through a process such as thick film screen printing or similar such processes.

FIGS. 9A and 9B show the various layers of the jaw member 520, seal plate assembly 523 and thermal cutter assembly 530 with parts separated (FIG. 9A) and in cross section (FIG. 9B). Once assembled with the various, above-identified components properly seated, layered or otherwise disposed, jaw member 520 is overmolded in one or two steps to secure the seal plate 523 assembly and outer jaw housing 522.

In embodiments, the end effector assembly 500 may be sprayed as a whole or along parts thereof with a material to create a thermal barrier between the thermal cutter assembly 530 and the surrounding jaw components. Thermal materials such as YSZ or other materials that have a low-thermal conductivity, and that are highly electrically insulative may be utilized for this purpose.

Jaw member 510 may be manufactured in a similar fashion to jaw member 520 as described above and may include an opposing thermal cutter assembly (not shown) which, together with thermal cutter 530, would cooperate to cut tissue disposed between jaw members 510, 520. Alternatively, jaw member 510 may simply include a thermal insulating material or substrate disposed in vertical registration with the thermal cutter assembly 530 of jaw member 520. More particularly, jaw member 510 includes a seal plate 513a welded atop seal plate 513b such that upper tissue contacting surface 514 extends on either side thereof defining channel 511 therebetween. A series of stop members 515 may be disposed atop tissue contacting surface 514 for defining a gap distance between opposing jaw members, e.g., jaw members 110, 120, during sealing. The stop members 515 may be applied as part of a thermal spraying process.

A substrate 509 (e.g., YSZ or some other type of ceramic or other material with a low thermal conductivity) may be thermally sprayed or masked atop seal plate 513b within channel 511 and is configured to thermally insulate jaw member 510 from the thermal cutter assembly 530 during activation thereof.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A jaw member for an end effector assembly of a vessel sealing instrument, comprising:
a seal plate assembly including first and second seal plates joined atop one another, the second seal plate defining a channel extending from a proximal to a distal end thereof;
a thermal cutter assembly including:
a substrate disposed within the channel and extending from the proximal to distal end of the second seal plate;
an insulator disposed atop the substrate and configured to extend therealong;
a resistive element disposed atop the insulator and configured to generate heat upon activation thereof; and
an encapsulant configured to electrically insulate the resistive element and thermally conduct heat from the resistive element, such that, upon activation thereof, tissue disposed within the end effector assembly is cut along the resistive element.

2. The jaw member according to claim 1, wherein the substrate is made from a material having a low thermal conductivity.

3. The jaw member according to claim 1, wherein the substrate is disposed within the channel utilizing a thermal spraying or deposition process.

4. The jaw member according to claim 1, wherein the insulator is disposed atop the substrate using a deposition process.

5. The jaw member according to claim 1, wherein the insulator is made from a material having a high coefficient of thermal conductivity but low electrical conductivity.

6. The jaw member according to claim 4, wherein the insulator is treated after the deposition process to facilitate adhesion of the resistive element thereon.

7. The jaw member according to claim 1, wherein opposing ends of the resistive element are configured to connect to a pair of conductive pads disposed at a proximal end of the insulator.

8. The jaw member according to claim 1, wherein the encapsulant is made from an electrically insulative, highly thermally conductive material.

9. An end effector assembly of a vessel sealing instrument, comprising:
first and second jaw members movable between a spaced apart position and an approximated position for sealing and cutting tissue, the first jaw member including:
a first seal plate assembly including first and second seal plates joined atop one another, the second seal plate defining a first channel extending from a proximal to a distal end thereof;
a thermal cutter assembly including:
a first substrate disposed within the first channel and extending from the proximal to distal end of the second seal plate;
an insulator disposed atop the first substrate and configured to extend therealong;
a resistive element disposed atop the insulator and configured to generate heat upon activation thereof; and
an encapsulant configured to electrically insulate the resistive element and thermally conduct heat from the resistive element, such that, upon activation thereof, tissue disposed between opposing first and second jaw members of the end effector assembly is cut along the resistive element; and
the second seal plate including a second seal plate assembly including first and second seal plates joined atop one another, the second seal plate defining a second channel extending from a proximal to a distal end thereof configured to receive a second substrate therein, wherein the second substrate opposes the thermal cutter assembly of the first jaw member when the first and second jaw members are moved to the approximated position.

10. The end effector assembly of a vessel sealing instrument according to claim 9, wherein the first and second substrates are made from materials having a low thermal conductivity.

11. The end effector assembly of a vessel sealing instrument according to claim 9, wherein at least one of the first or second substrates is disposed within a respective first and second channel utilizing a thermal spraying or deposition process.

12. The end effector assembly of a vessel sealing instrument according to claim 9, wherein the insulator is disposed atop the first substrate using a deposition process.

13. The end effector assembly of a vessel sealing instrument according to claim 9, wherein the insulator is made from a material having a high coefficient of thermal conductivity but low electrical conductivity.

14. The end effector assembly of a vessel sealing instrument according to claim 12, wherein the insulator is treated after the deposition process to facilitate adhesion of the resistive element thereon.

15. The end effector assembly of a vessel sealing instrument according to claim 9, wherein opposing ends of the resistive element are configured to connect to a pair of conductive pads disposed at a proximal end of the insulator.

16. The end effector assembly of a vessel sealing instrument according to claim 9, wherein the encapsulant is made from an electrically insulative, highly thermally conductive material.

17. A method of manufacturing a jaw member of an end effector assembly of a vessel sealing instrument, comprising:
joining first and second seal plates to form a seal plate assembly, the second seal plate defining a channel extending from a proximal to a distal end thereof, the channel configured to support a thermal cutter assembly formed therein via:
disposing a substrate within the channel extending from the proximal to distal end of the second seal plate;
disposing an insulator atop the substrate and extending the insulator therealong;
disposing a resistive element atop the insulator, the resistive element configured to generate heat upon activation thereof; and
encapsulating the resistive element with an electrically insulative, thermally conductive material, such that, upon activation of the resistive element, the electrically insulative, thermally conductive material heats to a temperature to cut tissue.

18. The method of manufacturing a jaw member of an end effector assembly of a vessel sealing instrument according to claim 17, wherein the substrate is made from a material having a low thermal conductivity and is disposed within the channel by a thermal spraying or masking process.

19. The method of manufacturing a jaw member of an end effector assembly of a vessel sealing instrument according to claim 17, wherein the insulator is made from a material having a high coefficient of thermal conductivity but low electrical conductivity and is disposed atop the substrate using a deposition process.

20. The method of manufacturing a jaw member of an end effector assembly of a vessel sealing instrument according to claim 17, wherein disposing the resistive element atop the insulator includes at least one of sputtering or thick film printing.
